(19) United States
(12) Patent Application Publication
(10) Pub. No.: US 2022/0387635 A1
(43) Pub. Date: Dec. 8, 2022

(54) PET IMAGING OF CANCEROUS CELLS USING 18F-FLUOROACETATE

(71) Applicant: Board of Regents, The University of Texas System, Austin, TX (US)

(72) Inventor: Amer NAJJAR, Houston, TX (US)

(73) Assignee: Board of Regents, The University of Texas System, Austin, TX (US)

(21) Appl. No.: 17/774,136

(22) PCT Filed: Nov. 4, 2020
(86) PCT No.: PCT/US2020/058839

§ 371 Date: May 3, 2022

Related U.S. Application Data
(60) Provisional application No. 62/930,429, filed on Nov. 4, 2019.

(51) Int. Cl. A61K 51/04

(52) U.S. Cl. CPC A61K 51/04

(57) ABSTRACT

The present disclosure provides methods of imaging cancerous cells in a subject, wherein the cancerous cells are localized to the skeletal system or central nervous system of the subject, the method comprising administering to the subject an effective amount of 18F-fluoroacetate, detecting a first signal emitted by 18F-fluoroacetate, and generating an image representative of the location and/or amount of the first signal to image the cancerous cells. In some embodiments, the methods further comprising diagnosing, prognosing, staging, and/or monitoring the progression of a disease or disorder, such as acute lymphoblastic leukemia and/or leptomeningeal disease.

### REFERENCE TO RELATED APPLICATIONS

This application claims the benefit of priority to U.S. Provisional Application No. 62/930,429, filed on Nov. 4, 2019, the entire contents of which is hereby incorporated by reference.

### GOVERNMENT SUPPORT CLAUSE

This invention was made with government support under grant number R01 CA163587 awarded by the National Institutes of Health. The government has certain rights in the invention.

### BACKGROUND

### I. Field

The present disclosure relates to the fields of radiology, imaging, and diagnostics. More specifically, it relates to methods of imaging cancerous cells using ¹⁸F-fluoroacetate, such as acute lymphoblastic leukemia cells (ALL).

### II. Description of Related Art

Leptomeningeal disease (LMD) is a late stage malignancy with poor prognosis characterized by the migration of malignant cells from a primary tumor site into the cerebrospinal fluid (CSF) and engrafting throughout the subarachnoid space of the leptomeninges of the brain and spinal cord.

The most effective means of treating leptomeningeal disease entails direct administration of chemotherapy into the CSF by lumbar puncture or via a ventricular access device called an Ommaya. Early therapeutic intervention with radiotherapy and chemotherapy have been shown to improve median survival from 4-6 weeks to 3-6 months, emphasizing the importance of early detection in facilitating earlier therapeutic intervention to improve survival.

Early detection and monitoring of LMD is critical to improving therapeutic outcomes and managing treatments. However, initial diagnosis of LMD and therapy response assessments remain challenging due to a lack of standardization and consensus on practices and methods. Furthermore, LMD can be easily overlooked without appropriate diagnostic tools and imaging (Sekhar et al., 2017). The Response Assessment of Neuro-Oncology (RANO) has proposed a combination of neurological examination, CSF cytology, flow cytometry, and radiographic evaluation by contrast-enhanced neuroaxis (brain and spine) magnetic resonance imaging to assess therapeutic responses. However, these assessments often suffer from limitations including difficulty associating ambiguous symptoms with LMD, high false negative or false positive rates due to poor selectivity, or difficulty in detecting small and/or non-solid tumors by magnetic resonance imaging (MRI).

Imaging of LMD by positron-emission tomography (PET) has been utilized with limited success using ¹⁸F-fluorodeoxyglucose (FDG) and amino acid (¹¹C-methionine) radiotracers (Barp et al., 2016; Fonti et al., 2016; Padma et al., 2001; Shah et al., 2007). This imaging modality is based on elevated tumor cell metabolism of glucose or amino acid uptake for biomass generation. Practical implementation of PET imaging for LMD diagnosis, however, has not be widely adopted due to limitations related to specificity and sensitivity of detection. Thus, there is a need for alternative methods of imaging tumors, particularly within hypoxic environments, such as bone marrow and meninges, where ALL cells thrive.

### SUMMARY

In some aspects, the present disclosure provides methods of imaging cancerous cells in a subject, wherein the cancerous cells are localized to the skeletal system or central nervous system of the subject, the method comprising: (a) administering to the subject an effective amount of ¹⁸F-fluoroacetate; (b) detecting a first signal emitted by ¹⁸F-fluoroacetate; and (c) generating an image representative of the location and/or amount of the first signal to image the cancerous cells. In some embodiments, the cancerous cells are engrafted in the skeletal system or central nervous system of the subject. In some embodiments, the cancerous cells are blood, brain, lung, breast, or skin cancer cells. In some embodiments, the cancerous cells are blood cancer cells. In further embodiments, the blood cancer cells are leukemia cells. In still further embodiments, the leukemia cells are acute lymphoblastic leukemia (ALL) cells.

In some embodiments, the subject is a mammal, such as a human. In some embodiments, the first signal is emitted by ¹⁸F-fluoroacetate in the skeletal system or central nervous system. In some embodiments, detecting the first signal is performed using positron emission tomography (PET). In some embodiments, the methods further comprise detecting a second signal. In some embodiments, detecting the second signal is performed using magnetic resonance imaging (MRI), computed tomography (CT), or single photon emission computed tomography (SPECT). In some embodiments, detecting the second signal is performed in conjunction with detecting the first signal. In some embodiments, detecting the first signal and detecting the second signal are performed using PET/CT or PET/MRI.

In some embodiments, the cancerous cells are localized to the skeletal system of the subject. In further embodiments, the cancerous cells are localized to the bone marrow. In other embodiments, the cancerous cells are localized to the central nervous system of the subject. In further embodiments, the cancerous cells are localized to one or more meninx. In still further embodiments, the cancerous cells are localized to the arachnoid mater or pia mater.

In some embodiments, the methods further comprise diagnosing, prognosing, staging, or monitoring the progression of a disease or disorder. In some embodiments, the disease or disorder is cancer. In further embodiments, the cancer is a blood, brain, lung, breast, or skin cancer. In still further embodiments, the cancer is a blood cancer. In yet further embodiments, the cancer is leukemia. In further embodiments, the leukemia is acute lymphoblastic leukemia. In some embodiments, the disease or disorder is leptomeningeal disease.

In some embodiments, the ¹⁸F-fluoroacetate is formulated as a pharmaceutical composition. In some embodiments, the ¹⁸F-fluoroacetate is formulated for administration via injection. In some embodiments, administering is via injection. In some embodiments, detecting the first signal is performed less than 5 hours after administering. In further embodiments, detecting the first signal is performed less than 3 hours after administering. In still further embodiments, detecting the first signal is performed less than 2 hours after administering. In yet further embodiments, detecting the first signal is performed about 1 hour after administering.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows NALM-6 ALL cells exhibit preferential metabolic uptake of [³H]-fluoroacetate over [³H]-glucose. Cell uptake studies were performed in the presence of [³H]-flouroacetate, [³H]-glucose, and [¹⁴C]-thymidine. Uptake levels were measure at 30, 60, and 120-minute intervals and expressed as a ratio of cellular retention versus concentration in surrounding media (cell:media ratio). Uptake rates were determined as a function of the slope of linear trendline. [³H]-flouroacetate uptake rates were higher (dotted line; 0.053 min⁻¹) compared to glucose (dashed line; 0.028 min-1) and thymidine (solid line; 0.024 min⁻¹).

FIGS. 2A & 2B show detection of leptomeningeal disease by fluorescence and immunohistological staining. NSG mice systemically injected with NALM-6 acute lymphoblastic leukemia cells expressing firefly luciferase and GFP (HALM-6 GFP-ffLuc) developed systemic disease distributed throughout the bone and the central nervous system. (FIG. 2A) Fluorescence imaging of whole brain and spine reveals localization of tumor cells throughout the spine and within the meninges of the brain. (FIG. 2B) Immunohistological staining for human CD19 revealed specific localization of tumor cells within the meninges in the forebrain, midbrain, brainstem, and spinal cord. Perivascular tumor cells (arrows) were detected suggesting vessel-mediated infiltration of meningeal tissue.

FIGS. 3A-3C show detection of leptomeningeal disease by bioluminescence imaging (BLI), PET-CT and combined PET-MM. NSG mice were injected intravenously with 2×10⁴ NALM-6 GFP-ffLuc cells. (FIG. 3A) Three weeks post-injection, the mice developed systemic disease localized throughout the bone and the central nervous system as demonstrated by BLI. (FIG. 3B) The mice were imaged by [¹⁸F]-fluoroacetate PET/CT to detect leptomeningeal infiltration of the tumor as illustrated by co-registration of the PET signal with brain and spinal cord (Left: mouse injected with NALM-6 ffLuc-GFP cells; Right: control mouse, no tumor cells injected). (FIG. 3C) PET/MRI of normal (control) mouse (right) and mouse bearing systemic NALM-6 tumor (left) reveals specific uptake of [¹⁸F]-fluoroacetate within the soft-tissue component of the brain and spinal cord, consistent with the presence of tumor cells throughout the leptomeningeal compartments.

FIG. 4 shows the time-activity curve of [¹⁸F]-fluoroacetate distribution, derived from 1-hour dynamic PET scans, within regions-of-interest in the brain and spinal cord. The data demonstrates time-dependent resolution and preferential accumulation of the radiotracer in regions of NALM-6 tumor within the central nervous system. Lower baseline levels of [′⁸F]-fluoroacetate uptake are detected in comparable regions of the brain and spinal cord of control mouse.

### DESCRIPTION OF ILLUSTRATIVE EMBODIMENTS

The present disclosure provides inter alia methods of imaging cancerous cells using ¹⁸F-fluoroacetate, such as acute lymphoblastic leukemia cells. The methods of the present disclosure may further comprise diagnosing, prognosing, staging, or a monitoring a disease or disorder, such as leptomeningeal disease.

### I. ¹⁸F-Fluoroacetate

As used herein, “¹⁸F-fluoroacetate” refers to a compound of the formula:

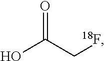

or a pharmaceutically acceptable salt thereof, such as sodium ¹⁸F-fluoroacetate (i.e., NaOC(O)CH₂¹⁸F). While PET with ¹¹C-acetate exhibits sensitivity for several cancers, ¹⁸F-fluoroacetate has a longer radioactive half-life (¹⁸F t_{1/2}=110 min). ¹⁸F-fluoroacetate may also have the advantage that it may be more efficacious than, be less toxic than, be longer acting than, be more potent than, produce fewer side effects than, be more easily absorbed than, and/or have a better pharmacokinetic profile (e.g., higher bioavailability and/or lower clearance) than, and/or have other useful radiological, physical, or chemical advantages over, compounds known in the prior art for use in the indications stated herein.

### II. Leptomeningeal Disease

Leptomeningeal disease (LMD) is the third most common metastatic complication involving the CNS and is becoming increasingly prevalent as improvements in therapy are prolonging patient survival (Chamberlain, 2010). Leptomeningeal metastases occur in 1-5% of patients with solid tumors (carcinomatous meningitis), 1-2% of brain tumor patients, and has the highest incidence (5-15%) among patients with leukemia (leukemic meningitis) and lymphoma (lymphomatous meningitis) (Chamberlain, 2010). Although LMD is most often associated with disseminated progressive cancer, it can also be present in patients during remission or even detected at initial diagnosis.

Early detection and monitoring of LMD is critical to improving therapeutic outcomes and managing treatments. However, initial diagnosis of LMD and therapy response assessments remain challenging due to a lack of standardization and consensus on practices and methods. Furthermore, LMD can be easily overlooked without appropriate diagnostic tools and imaging (Sekhar et al., 2017). The Response Assessment of Neuro-Oncology (RANO) has proposed a combination of neurological examination, CSF cytology, flow cytometry, and radiographic evaluation by contrast-enhanced neuroaxis (brain and spine) magnetic resonance imaging to assess therapeutic responses.

Neurological assessments to detect progressive LMD are based on changes in cognitive and motor functions such as gait, strength, sensation, vision, hearing or behavior. Patients may also present with facial weakness and radiating pain. However, symptoms are often ambiguous and difficult to associate with LMD (Barp et al., 2016). These assessment domains may also be impacted by extraneous factors such as coexistent brain metastases and treatment-related toxicities. Distinction between LMD- and treatment-related neurological deficits can be made based on duration of symptoms, where LMD-related deficit tend to be irreversible compared to the transient nature of treatment-related deficiencies.

Cytology offers a qualitative assessment of LMD, with binary assessment classifications (negative or positive) of all sampling sites. However, CSF cytology false negative rates approach 50% due to poor sensitivity (Jung et al., 2013). Reliability of CSF cytology is dependent on multiple factors including volume of collection, sampling site, processing speed as cellular viability is susceptible to manipulation and processing after collection (Quinten et al., 2014; Chamberlain et al., 2009; Aune et al., 2004).

CSF flow cytometry is qualitative and offers higher sensitivity as it relies on quantitative automation (Nuckel et al., 2006). CSF flow is more informative in patients with hematological malignancies as a prognostic factor indicating CNS relapse (Nuckel et al., 2006; Dux et al., 1991; Hegde et al., 2005). However, increased sensitivity can often result in false positive assessments, and interpretation should be regarded with caution in patients without hematological malignancies (Nuckel et al., 2006; Glantz et al., 1998; Bromberg et al., 2007).

Neuroaxis magnetic resonance imaging (MRI) plays a critical role in LMD diagnosis. LMD MM presents as enhancement of the leptomeninges of the brain and spine (Freilich et al., 1995; Chamberlain and Kormanik, 1997; Clarke et al., 2010; Pauls et al., 2012) and is often confirmed by CSF flow studies with radioisotopes to identify regions of obstruction and cytological detection of tumor cells in the CSF. However, MRI is often subjective due to the disseminated nature of the disease, and quantitative assessment by MM is not practical as radiographic lesions present as small and geometrically complex regions (Pauls et al., 2012; Smirniotopoulos et al., 2007). Thus, quantitative imaging is limited to lesions >5 mm due to variabilities of MRI slice positioning/spacing and contrast conspicuity. Imaging abnormalities are detected in 70-80% of patients with leptomeningeal disease and is more often detected in with solid tumors versus hematological malignancies. MRI is only able to detect tumors in 16-20% of patients with hematological malignancies (Chamberlain, 2013).

MM also has low specificity as contrast enhancement is not always consistent with metastasis. Baseline neuroaxis MRI is thus recommended. In some cases, surgery or intracranial hypotension following lumbar puncture can results in false positive meningeal enhancement that mimics LMD (Pauls et al., 2012; Wesley et al., 2016). Normal MRI in patients with LMD is not uncommon and, as such, neuroaxis MRI is more informative when positive. Variations in gadolinium formulation and dose, infusion parameters, and elapsed time are other factors that may introduce variability in the interpretation of MM. In some embodiments, the present disclosure provides methods of diagnosing, prognosing, staging, and/or monitoring the progression of a disease or disorder, such as acute lymphoblastic leukemia or leptomeningeal disease.

### III. Imaging

In some aspects, the present disclosure provides methods of imaging using the compounds and compositions of the present disclosure. In some embodiments, the imaging is positron-emission tomography. Positron emission tomography (PET) imaging is based on detecting two time-coincident high-energy photons from the emission of a positron-emitting radioisotope. PET imaging is unique in its very high sensitivity and accurate estimation of the in vivo concentration of the radiotracer. PET imaging has been widely adopted as an important clinical modality for oncological, cardiovascular, and neurological applications. PET imaging has also become an important tool in preclinical studies, particularly for investigating murine models of disease and other small-animal models. In some embodiments, the present disclosure methods of imaging using compounds comprising a radioisotope, such as ¹⁸F. In some embodiments, the methods comprise detecting signals emitted by ¹⁸F-fluoroacetate. In some embodiments, the signals are positrons emitted by the ¹⁸F radionuclide in ¹⁸F-fluoroacetate. In some embodiments, detecting the signals is performed using PET, SPECT, CT, MM, or a combination thereof, such as PET/CT or PET/MRI.

### IV. Formulations and Routes of Administration

In another aspect, for administration to a patient in need of diagnostic or prognostic evaluation, staging, and/or progression monitoring, radiopharmaceutical formulations (also referred to as radiopharmaceutical preparations, radiopharmaceutical compositions, or radiopharmaceutical products) comprise an effective amount of a radiopharmaceutical, i.e., ¹⁸F-fluoroacetate, formulated with one or more excipients and/or drug carriers appropriate to the indicated route of administration.

In some embodiments, the radiopharmaceutical is formulated in a manner amenable for diagnostic or prognostic evaluation, staging, and/or progression monitoring of human and/or veterinary subjects. In some embodiments, formulation comprises admixing or combining the radiopharmaceutical with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, the radiopharmaceutical may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the radiopharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Radiopharmaceutical formulations may be administered by a variety of methods, such as by injection (e.g., subcutaneous, intravenous, and intraperitoneal). Depending on the route of administration, the radiopharmaceutical may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes. The radiopharmaceutical may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Radiopharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of radiopharmaceutical calculated to produce the desired imaging effect in association with the required pharmaceutical carrier. In some embodiments, the specification for the dosage unit forms of the disclosure are dictated by and directly dependent on (a) the unique characteristics of the radiopharmaceutical and the particular imaging effect to be achieved, and (b) the limitations inherent in the art of compounding such a radiopharmaceutical for the imaging of a patient. In some embodiments, active compounds are administered at an effective dosage sufficient for detection and production of a PET image in a patient. For example, the efficacy of a radiopharmaceutical can be evaluated in an animal model system that may be predictive of efficacy in imaging a human or another animal.

In some embodiments, an imaging agent comprising an isotope, such as a radioisotope, may be referred to as being “isotopically enriched.” An “isotopically enriched” composition refers to a composition comprising a percentage of one or more isotopes of an element that is more than the percentage (of such isotope) that occurs naturally. As an example, a composition that is isotopically enriched with a fluoride species may be “isotopically enriched” with fluorine-18 (¹⁸F). Thus, with regard to a plurality of compounds, when a particular atomic position is designated as ¹⁸F, it is to be understood that the abundance (or frequency) of ¹⁸F at that position (in the plurality) is greater, including substantially greater, than the natural abundance (or frequency) of ¹⁸F, which is essentially zero. In some embodiments, a fluorine designated as may have a minimum isotopic enrichment factor of about 0.001% (i.e., about 1 out of 10⁵ fluorine species is ¹⁸F), 0.002%, 0.003%, 0.004%, 0.005%, 0.006%, 0.007%, 0.008%, 0.009%, 0.01%, about 0.05%, about 0.1%, about 0.2%, about 0.3%, about 0.4%, about 0.5%, about 0.75%, about 1%, about 2%, about 3%, about 4%, about 5%, about 10%, about 15%, about 20%, about 30%, about 40%, about 50%, about 60%, about 70%, about 80%, about 90%, about 95%, or greater. The minimum isotopic enrichment factor, in some instances, may range from about 0.001% to about 1%. The isotopic enrichment of the compounds provided herein can be determined using conventional analytical methods known to one of ordinary skill in the art, including mass spectrometry and HPLC.

In some embodiments, the effective radioactivity dose range for the radiolabeled compound can be extrapolated from effective doses determined in animal studies for a variety of different animals. In some embodiments, the radiopharmaceutical is administered by intravenous injection, usually in saline solution, at a dose of between about 0.1 and about 50 mCi (and all combinations and subcombinations of dosage ranges and specific dosages therein), and as described below. Precise amounts of the radiopharmaceutical depend on the judgment of the practitioner and are specific to each individual. Imaging is performed using techniques well known to the ordinarily skilled artisan and/or as described herein.

The maximum desirable dose administered to a subject may be based on determining the amount of radiopharmaceutical that limits the radiation dose to about 5 rem to the critical organ (e.g., urinary bladder) and/or about 1 rem effective dose (ED) or lower, as will be understood by those of ordinary skill in the art. In some embodiments, the maximum desirable dose or total amount of radiopharmaceutical administered is between about 1 mCi and about 20 mCi. In some embodiments of the disclosure, the maximum desirable dose or total amount of radiopharmaceutical administered is between about 5 mCi and about 15 mCi. In some embodiments of the disclosure, the maximum desirable dose or total amount of radiopharmaceutical administered is between about 8 mCi and about 12 mCi. In some embodiments, a desirable dose may be less than or equal to about 15 mCi, less than or equal to about 14 mCi, less than or equal to about 13 mCi, less than or equal to about 12 mCi, less than or equal to about 11 mCi, or less than or equal to about 10 mCi over a period of time of up to about 10 minutes, about 30 minutes, about 1 hour, about 2 hours, about 6 hours, about 12 hours, about 24 hours, or about 48 hours.

In some embodiments, the total amount of radiopharmaceutical administered to a subject is between about 0.1 mCi and about 30 mCi, or between about 0.5 mCi and about 20 mCi. In some embodiments, the total amount of radiolabeled compound administered to a subject is less than or equal to about 50 mCi, less than or equal to about 40 mCi, less than or equal to about 30 mCi, less than or equal to about 20 mCi, less than or equal to about 18 mCi, less than or equal to about 16 mCi, less than or equal to about 15 mCi, less than or equal to about 14 mCi, less than or equal to about 13 mCi, less than or equal to about 12 mCi, less than or equal to about 10 mCi, less than or equal to about 8 mCi, less than or equal to about 6 mCi, less than or equal to about 4 mCi, less than or equal to about 2 mCi, less than or equal to about 1 mCi, or less than or equal to about 0.5 mCi. The total amount administered may be determine based on a single dose or multiple doses administered to a subject within a time period of up to or at least about 30 seconds, about 1 minute, about 10 minutes, about 30 minutes, about 1 hour, about 2 hours, about 6 hours, about 12 hours, about 24 hours, about 48 hours, or about 1 week.

In some embodiments, between about 0.1 and about 30 mCi of radiolabeled compound is administered to a subject, and a first period of image acquisition begins at the time of administration (e.g., injection) or begins at more than about 0 minutes, about 1 minute, about 2 minutes, about 3 minutes, about 4 minutes, about 5 minutes, prior to the administration of the radiolabeled compound. In some embodiments, the first imaging continues for at least about 5 minutes, about 10 minutes, about 15 minutes, about 30 minutes, about 45 minutes, about 60 minutes, about 75 minutes, about 90 minutes, about 105 minutes, about 120 minutes, or longer. Following the first period of imaging, the subject may undergo one or more additional imaging acquisition periods during up to about 1, about 2, about 3, about 4, about 5, about 6, or more hours after the administration of radiopharmaceutical. One or more additional image acquisition periods may have a duration of between about 3 and about 40 minutes, about 5 and about 30 minutes, about 7 and about 20 minutes, about 9 and about 15 minutes, and may be for about 10 minutes. The subject, in some embodiments, may return once, twice, or three or more times for additional imaging following the first injection of radiopharmaceutical wherein a second, third, or more, injections of radiopharmaceutical may be administered. A non-limiting example of an administration and image acquisition method for radiopharmaceutical compound for a subject comprises injection of between about 0.1 and about 30 mCi of radiopharmaceutical compound to the subject, with image acquisition starting less than about 10 minutes before the injection and continuing for about 60 minutes. In some embodiments, the subject undergoes first or additional image acquisition for about 10 minutes, or for about 20 minutes, or for about 30 minutes, or for about 40 minutes, or for about 50 minutes, or for about 60 minutes, at about one hour, or about two hours, or about 3 hours, or about 4 hours, and at about 4 hours, or about 5 hours, or about 6 hours, or about 7 hours, or about 8 hours, after the injection of the radiopharmaceutical.

Principles and techniques for radiopharmaceutical dosimetry are taught, for example, in Zanzonico, 2000, which is incorporated by reference herein. Other factors affecting the dose include the physical and clinical state of the patient, the route of administration, the intended goal of imaging, and the stability and toxicity of the particular radiopharmaceutical formulation. The actual dosage amount of a compound of the present disclosure or composition comprising a compound of the present disclosure administered to a patient may be determined by physical and physiological factors such as type of subject treated, age, sex, body weight, severity of condition, the type of disease being imaged, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

### V. Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, radiopharmaceutical agent, agent, radiologically active molecule, or a radiopharmaceutical) is the ingredient in a radiopharmaceutical drug that is radiologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” in the context of imaging a patient means that amount of the compound or composition which, when administered to the subject or patient, is sufficient to be detectable and thus suitable to generate an image, such as a PET image. “Effective amount,” “therapeutically effective amount” or “pharmaceutically effective amount” when used in the context of treatment, therapy, prevention or diagnosis, means that amount of the compound which, when administered to a subject or patient, is sufficient to effect such treatment, therapy, prevention, or diagnosis, respectively.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired radiological activity. Such salts include acid addition salts formed with inorganic acids such as hydrofluoric acid, hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient that is involved in carrying, delivering and/or transporting the ingredient or compound. Drug carriers may be used to improve the delivery and the effectiveness of drugs or diagnostic agents, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “radiopharmaceutical drug” (also referred to as a radiopharmaceutical, radiopharmaceutical preparation, radiopharmaceutical composition, radiopharmaceutical formulation, radiopharmaceutical product, or simply a drug, agent, or preparation) is a composition used to image, diagnose, prognose, stage, and/or monitor a disease or disorder, which comprises an active pharmaceutical ingredient (API) (defined above) and optionally contains one or more inactive ingredients, which are also referred to as excipients (defined above).

“Imaging” includes any technique and processing method of creating visual representations of the interior of a body for clinical analysis and medical intervention, as well as visual representation of the physiological or biochemical function of some organs or tissues.

“Positron emission tomography (PET)” is a nuclear imaging technology (also referred to as molecular imaging) that enables visualization of the fate of a radiopharmaceutical in deep tissues in real time in living subjects. PET detects pairs of annihilation photons emitted by a positron-emitting radionuclide incorporated into a small molecule, peptide, protein or nanoparticle that contains moieties that confer targeting capacity to the entity and visualized from inside a living subject. The radionuclide and targeted carrier together are called a radiopharmaceutical or radiotracer.

The term “unit dose” refers to a formulation of the radiopharmaceutical or radiopharmaceutical composition such that the formulation is prepared in a manner sufficient to provide a single diagnostically effective dose of the active ingredient to a patient in a single administration. Such unit dose formulations that may be used include but are not limited to a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VI. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Methods and Results

In vitro uptake of [³H]-FA, [³H]-glucose, and [¹⁴C]-thymidine. Radiotracer uptake studies were performed as described previously (Najjar et al., 2009). Briefly, 10⁷ NALM-6 were suspended in 6 mL of RPMI medium containing 10% FBS, [¹⁴C]thymidine (0.01 μCi/mL; Moravek, Brea, Calif., USA) and [³H]-fluoroacetate (0.1 μCi/mL; Moravek) or [¹⁴C]thymidine (0.01 μCi/mL and [³H]-glucose (2.5 μCi/mL). At time intervals of 30, 60, and 120 minutes, 1 mL aliquots were removed and the cells were pelleted in pre-weighed vials. A 50-4, aliquot of the supernatant was transferred to a pre-weighed scintillation tube. The pellets were weighed following removal of the remainder of the media and thoroughly resuspended in 0.1 mL of Soluene-350 (Perkin-Elmer, Boston, Mass.). Radioactive beta emissions of the media and cell pellets were measured within pre-calibrated energy ranges using a Packard Tri-Carb 3100TR scintillation counter to quantitate [³H]-FA, [³H]-glucose, and [¹⁴C]-thymidine uptake. The activity accumulation ratios of the cell pellets to the media ([disintegrations per minute/g cells]/[disintegrations per minute/g media]) were then determined and plotted.

Mouse ALL model. All animal studies were approved by IACUC at The University of Texas M.D. Anderson Cancer Center. NALM-6 ALL cells expressing green fluorescent protein-firefly luciferase (NALM-6 GFP-ffLuc; 1.5×10⁴ cells/100 μL saline) were injected intravenously into 8-week-old NOD.Cg-PrkdcscidIl2rgtmlwjl/SzJ mice. Systemic growth of the tumor was monitored weekly by bioluminescence imaging (BLI) performed using a Bruker system. Mice were injected with D-luciferin (2 mg/kg in PBS; Caliper Life Sciences, Hopkinton, Mass., USA), anesthetized using isoflurane (2%) in 98% oxygen, and placed in prone positions in the imaging chamber. Images were acquired for a 10-minute period and quantitated with regions of interest (ROI).

Synthesis of ¹⁸F-FA. Synthesis of ¹⁸F-FA was carried out at the Center of Advanced Biomedical Imaging (CABI) as previously described (Nishii et al., 2012).

PET imaging of ALL animal model. Mice were anesthetized and positioned in a secure prone position. An intravenous bolus injection of [³H]-fluoroacetate (1.9 MBq in 100 μL of saline) was administered via the tail vein. Following a 1-hour period, static PET images were acquired on an Albira PET/computed tomography (CT) system. PET images were reconstructed using ordered subset expectation maximization algorithms. Regional radioactivity concentrations of [³H]-fluoroacetate were determined within regions of interest (ROI) on reconstructed images. Radiotracer uptake levels were expressed as mean percent injected dose per gram (% ID/g)±SD.

Immunohistochemistry. Immunohistochemistry (IHC) was performed on brain and spinal tissue to confirm leptomeningeal presence of tumor cells. Whole brain and spinal tissue were collected following necropsy, fixed in formalin for 24 hours, and transferred to 70% ethanol for 48-72 hours. Sagittal cross-sections (10 μm) of the forebrain, midbrain, brain stem, and spinal cord were obtained and stained for human CD19 expression using anti-human CD19. Histological H&E staining was also performed for anatomical referencing.

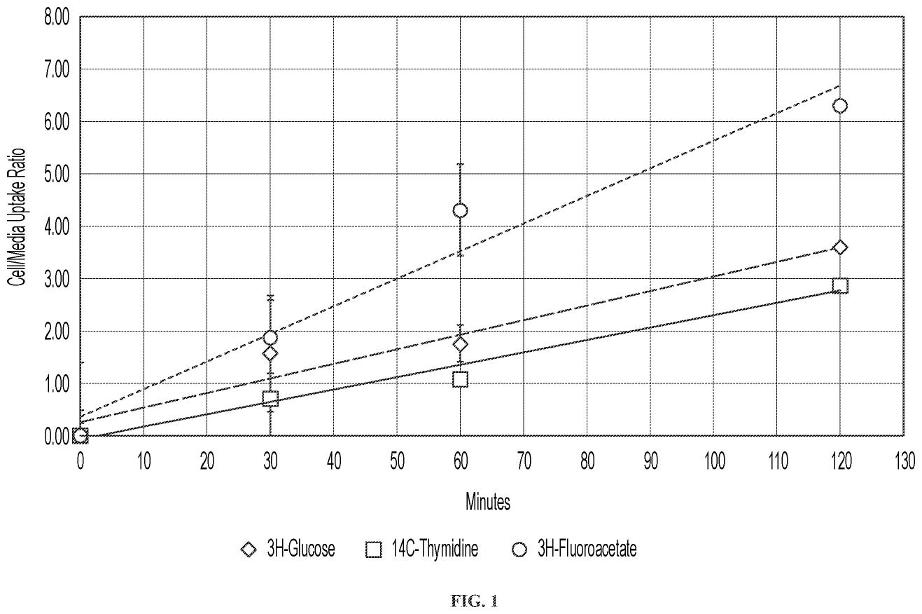

NALM-6 ALL cells exhibit preferential metabolic uptake of fluoro-acetate. Preferential engraftment and growth of leukemic cell within the fat saturated bone marrow environment suggests that propensity of these cells for fatty acid metabolism as a richer energy source. Accordingly, in vitro radiotracer uptake studies were performed to determine the metabolic preference of NALM-6 cells for uptake and retention of fluoroacetate and glucose. The accumulation [³H]-fluoroacetate and [³H]-glucose was measured and compared separate experiments (FIG. 1). Uptake levels were normalized to [¹⁴C]-thymidine levels for direct comparison. NALM-6 cells exhibited preferential uptake of [³H]-fluoroacetate at nearly twice the rate of [³H]-glucose (0.056 vs. 0.028 min⁻¹). The rates of [³H]-glucose and [¹⁴C]-thymidine were more closely matched at 0.028 and 0.024 min⁻¹, respectively.

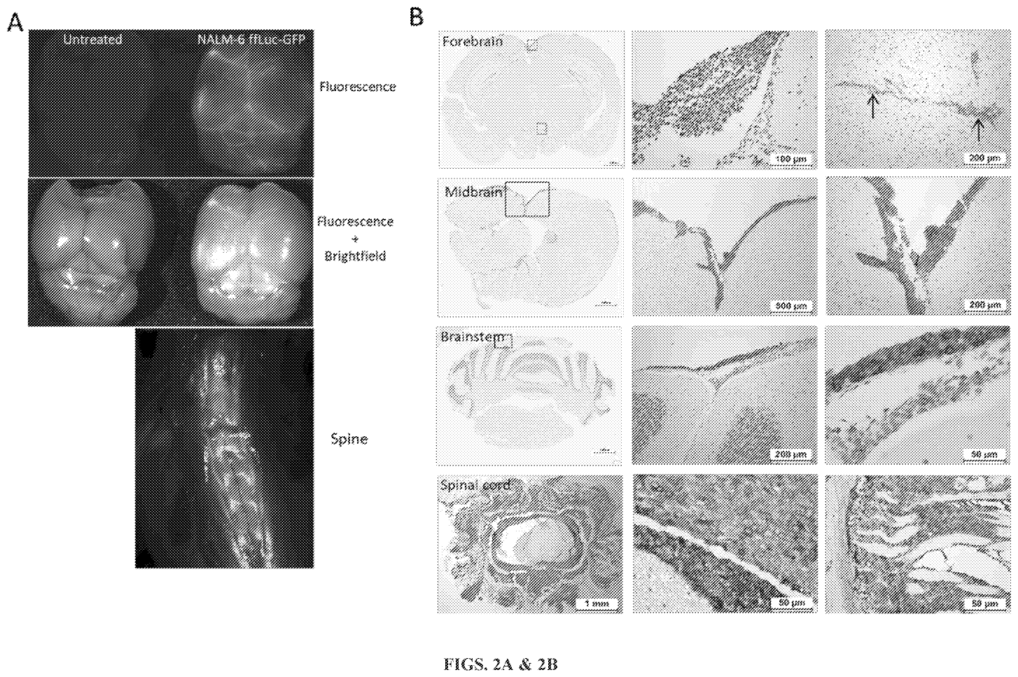

Mouse model of disseminated ALL and LMD. NSG mice injected intravenously with 2×10⁴ NALM-6 ALL cells expressing firefly luciferase and GFP (NALM-6 GFP-ffLuc) developed disseminated disease throughout the bone marrow and end-stage LMD 3-4 weeks post-injection. As shown in FIGS. 2A & 2B, localization of tumors cells throughout the skeletal system and central nervous system. The filamentous fluorescence pattern detected throughout the brain is consistent with the distribution of the meninges. Immunohistochemical staining for human CD19 expression confirmed the presence of ALL tumor cells throughout the meninges in the forebrain, midbrain, brainstem, and spinal cord. The presence of perivascular tumor cells (arrows) suggests vessel-mediated infiltration of meningeal tissue.

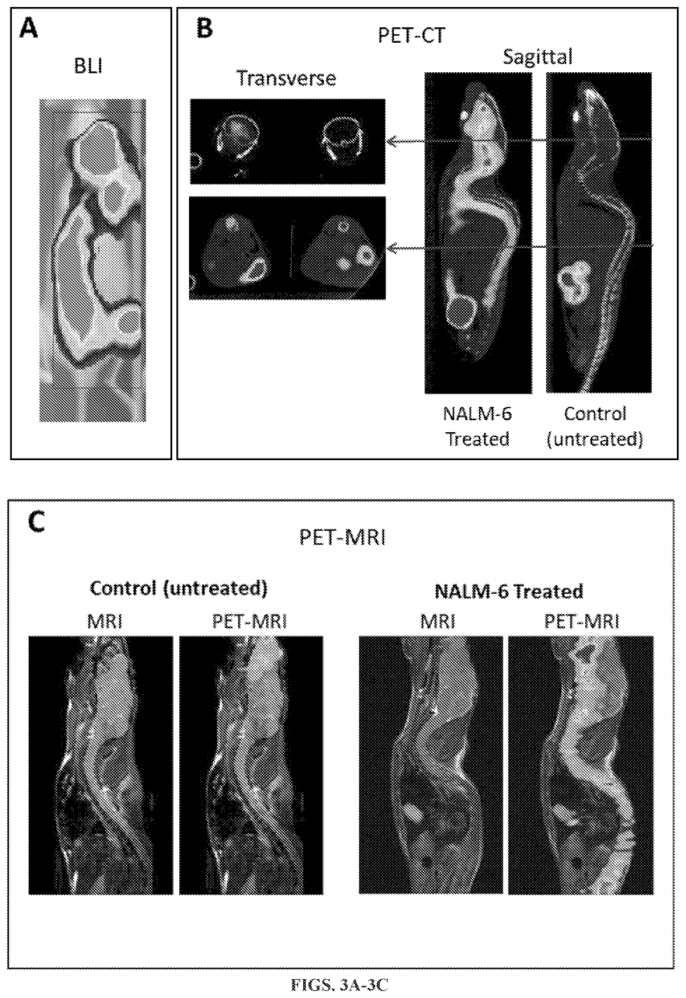

Detection of leptomeningeal disease by PET-CT and combined PET-MRI. Preferential in vitro uptake of [³H]-fluoroacetate prompted evaluation of in vivo uptake by PET imaging in the ALL mouse model (FIGS. 3A-3C). Bioluminescence imaging prior to PET confirmed the presence of disseminated disease throughout the skeletal system (FIG. 3A). The mice were subsequently imaged by [¹⁸F]-fluoroacetate PET-CT, which confirmed elevated uptake of the radiotracer throughout the central nervous system compared to untreated control (FIG. 3B). Combined PET-MRI was subsequently performed to precisely establish the presence of PET signal within the brain and spinal cord and detect leptomeningeal infiltration of the tumor (FIG. 3C).

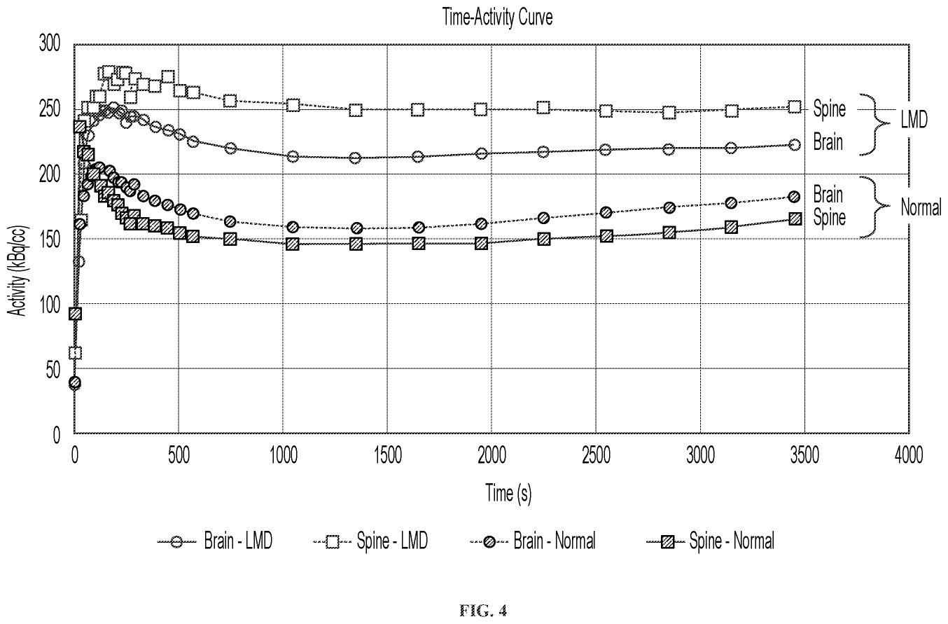

Time-activity curve of [¹⁸F]-fluoroacetate biodistribution within the brain and spinal cord. A dynamic PET scan was used to determine the time-dependent distribution [¹⁸F]-fluoroacetate within regions-of-interest (ROI) in the brains and spinal cords of normal (control) and NALM-6 tumor-bearing (LMD) mice. The time-activity curve reveals preferential uptake of the radiotracer consistent with the presence of disease (FIG. 4). Mice bearing systemic NALM-6 tumors exhibited higher baseline levels of [¹⁸F]-fluoroacetate uptake within the brain and spinal cord. In contrast, normal mice exhibited lower baseline levels of radiotracer uptake within the brain and spinal cord.

All of the compounds, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the disclosure. All variations and modifications apparent to those skilled in the art are deemed to be within the spirit, scope, and concept of the disclosure as defined by the appended claims.

### VII. References

The following references to the extent that they provide exemplary procedural or other details supplementary to those set forth herein, are specifically incorporated herein by reference.
Anderson, Practical Process Research & Development— A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB 22(3):659-661, 2008.
Smith, March's Advanced Organic Chemistry: Reactions, Mechanisms, and Structure, 7^{th} Ed., Wiley, 2013.
Aune et al., Am. J. Clin. Pathol., 121:690-700, 2004.
Barp et al., J. Neuro-oncol., 126:205-207, 2016.
Bromberg et al., Neurology, 68:1674-1679, 2007.
Chamberlain and Kormanik, Arch. Neurol., 54:1364-1368, 1997.
Chamberlain et al., Semin. Oncol., 36:S35-45, 2009.
Chamberlain, CNS Oncol., 2:121-128, 2013.
Chamberlain, Curr. Opin. Oncol., 22:627-635, 2010.
Clarke et al., Neurology, 74:1449-1454, 2010.
Dux et al., Cytometry, 12:422-428, 1991.
Fonti et al., Clin. Nuclear Med., 41:169-172, 2016.
Freilich et al. Ann. Neurol., 38:51-57, 1995.
Glantz et al., Cancer, 82:733-739, 1998.
Hegde et al., Blood, 105:496-502, 2005.
Jung et al., Support. Care Cancer, 21:3379-3386, 2013.
Najjar et al., J. Nuclear Med., 50:409-416, 2009.
Nishii et al., Mol. Imaging Biol., 14:213-224, 2012.
Nuckel et al., Clin. Lab. Haematol., 28:22-29, 2006.
Padma et al., J. Neuro-oncol., 55:39-44, 2001.
Pauls et al., Eur. J. Radiol., 81:974-978, 2012.
Quinten et al., Cancer, 120:302-311, 2014.
Sekhar et al., J. Royal College Physicians Edinburgh, 47:351-352, 2017.
Shah et al., Indian J. Cancer, 44:115-118, 2007.
Smirniotopoulos et al., Radiographics, 27:525-551, 2007.
Wesley et al., Neurology Clin. Practice, 6:315-320, 2016.
Zanzonico, J. Nuclear Med., 41(2):297-308, 2000.

What is claimed is:

1. A method of imaging cancerous cells in a subject, wherein the cancerous cells are localized to the skeletal system or central nervous system of the subject, the method comprising:
(a) administering to the subject an effective amount of ¹⁸F-fluoroacetate;
(b) detecting a first signal emitted by ¹⁸F-fluoroacetate; and
(c) generating an image representative of the location and/or amount of the first signal to image the cancerous cells.

2. The method of claim 1, wherein the cancerous cells are engrafted in the skeletal system or central nervous system of the subject.

3. The method of claim 1, wherein the cancerous cells are blood, brain, lung, breast, or skin cancer cells.

4. The method of either claim 1 or claim 3, wherein the cancerous cells are blood cancer cells.

5. The method of claim 4, wherein the blood cancer cells are leukemia cells.

6. The method of claim 5, wherein the leukemia cells are acute lymphoblastic leukemia (ALL) cells.

7. The method according to any one of claims 1-6, wherein the subject a mammal.

8. The method of claim 7, wherein the mammal is a human.

9. The method according to any one of claims 1-8, wherein the first signal is emitted by ¹⁸F-fluoroacetate in the skeletal system or central nervous system.

10. The method according to any one of claims 1-9, wherein detecting the first signal is performed using positron emission tomography (PET).

11. The method of claim 10, wherein the method further comprises detecting a second signal.

12. The method of claim 11, wherein detecting the second signal is performed using magnetic resonance imaging (MRI), computed tomography (CT), or single photon emission computed tomography (SPECT).

13. The method of either claim 11 or claim 12, wherein detecting the second signal is performed in conjunction with detecting the first signal.

14. The method of claim 13, wherein detecting the first signal and detecting the second signal are performed using PET/CT or PET/MRI.

15. The method according to any one of claims 1-14, wherein the cancerous cells are localized to the skeletal system of the subject.

16. The method of claim 15, wherein the cancerous cells are localized to the bone marrow.

17. The method according to any one of claims 1-14, wherein the cancerous cells are localized to the central nervous system of the subject.

18. The method of claim 17, wherein the cancerous cells are localized to one or more meninx.

19. The method of claim 18, wherein the cancerous cells are localized to the arachnoid mater or pia mater.

20. The method according to any one of claims 1-19, wherein the method further comprises diagnosing, prognosing, staging, or monitoring the progression of a disease or disorder.

21. The method of claim 20, wherein the disease or disorder is cancer.

22. The method of claim 21, wherein the cancer is a blood, brain, lung, breast, or skin cancer.

23. The method of claim 22, wherein the cancer is a blood cancer.

24. The method of claim 23, wherein the cancer is leukemia.

25. The method of claim 24, wherein the leukemia is acute lymphoblastic leukemia.

26. The method of claim 20, wherein the disease or disorder is leptomeningeal disease.

27. The method according to any one of claims 1-26, wherein the ¹⁸F-fluoroacetate is formulated as a pharmaceutical composition.

28. The method according to any one of claims 1-27, wherein the ¹⁸F-fluoroacetate is formulated for administration via injection.

29. The method according to any one of claims 1-28, wherein administering is via injection.

30. The method according to any one of claims 1-29, wherein detecting the first signal is performed less than 5 hours after administering.

31. The method of claim 30, wherein detecting the first signal is performed less than 3 hours after administering.

32. The method of claim 31, wherein detecting the first signal is performed less than 2 hours after administering.

33. The method of claim 32, wherein detecting the first signal is performed about 1 hour after administering.
